(19) United States
(12) Patent Application Publication
(10) Pub. No.: US 2003/0077322 A1
(43) Pub. Date: Apr. 24, 2003

(54) Solid dispersion system of pranlukast with improved dissolution and method for preparing the same

(76) Inventor: Sang Deuk Lee, Seoul (KR)

Correspondence Address: HESLIN ROTHENBERG FARLEY & MESITI PC, 5 COLUMBIA CIRCLE, ALBANY, NY, 12203, US

(21) Appl. No.: 10/300,299

(22) Filed: Nov. 19, 2002

Related U.S. Application Data
(63) Continuation of application No. PCT/KR01/00804, filed on May 17, 2001.

(30) Foreign Application Priority Data
May 20, 2000 (KR) 2000-27237

(51) Int. Cl. A61K 9/48; A61K 9/20; A61K 9/14

(52) U.S. Cl. 424/465; 424/452; 424/488

(57) ABSTRACT

A pharmaceutical composition of pranlukast having improved bioavailability is disclosed. The composition is formulated as a solid dispersion, preferably for oral administration. In one embodiment, the invention comprises an amount of pranlukast uniformly dispersed in an inert polymer carrier comprising at least one of hydroxypropylmethylcellulose or hydroxypropylcellulose. In another embodiment, the composition further comprises an amount of hydroxypropylmethylcellulose phthalate-50. A method for preparing the composition, an oral formulation comprising the composition, and methods of treating bronchial asthma and allergic rhinitis with the composition are also disclosed.

### CROSS REFERENCE TO RELATED APPLICATIONS

This application is a continuation of International Patent Application PCT/KR01/00804, filed May 17, 2001 and claims priority from Korean Patent Application Number 2000-27237, filed May 20, 2000. The entire content of the prior applications is incorporated herein by reference.

### FIELD OF THE INVENTION

The present invention relates to a pharmaceutical composition of pranlukast, a drug having very low water solubility, a method for preparing the composition, an oral formulation comprising the composition, and methods of treating bronchial asthma and allergic rhinitis with the composition.

### BACKGROUND OF THE INVENTION

4-oxo-8-[4-(4-phenylbutoxy)benzoyl-amino]-2-(tetrazol-5-yl)-4H-1-benzopyran hemihydrate, commonly known as “pranlukast” (trade name, “ONON™”, Ono Pharmaceutical Company, Ltd., Osaka, Japan/Schering-Plough Corporation, Madison, N.J. USA), is a potent leukotriene antagonist, especially with respect to leukotrienes C4(LTC4) and D4(LTD4). It is known to have efficacy as a therapeutic agent for bronchial asthma and allergic rhinitis, and is expected to have activity as a therapeutic agent for treating allergic shock and various allergic inflammatory responses, thus having a wide range of applications.

Unfortunately, however, pranlukast is only minimally water soluble, and consequently, its bioavailability upon oral administration is very low. Thus, in order to provide an effective oral dose, large amounts of the drug must be administered, which, of course, greatly increases the cost per dose.

Accordingly, a formulation comprising a pharmaceutical composition of pranlukast that has improved bioavailability upon oral administration would be highly desirable. To date, however, the present inventor is aware of no such formulation.

Examples of known pharmaceutical compositions of pranlukast are described in PCT Published International Patent Application WO 96/41628, which discloses granules containing pranlukast, a manufacturing method thereof and a method for lowering the cohesiveness of pranlukast; PCT Published International Patent Application WO 99/04790, which discloses an aqueous pharmaceutical composition containing a benzopyran derivative as a major component; and Laid-Open Japanese Patent Pyung 8-73353, which discloses a pharmaceutical preparation comprising pranlukast and polyvinylpyrrolidone or β-cyclodextrin. In addition, published studies have described, for example, a pranlukast powder aerosol that utilizes surface modification to provide improved efficiency upon inhalation. Pharmaceutical Research, 15:1748-1752 (1998). Each of these formulations, however, differs significantly from the present invention, which provides a pharmaceutical composition wherein the active component, pranlukast, is uniformly dispersed in a solid state inert polymer carrier. This formulation provides vastly improved oral bioavailability due to the improved dissolution of pranlukast in the composition.

WO 96/41628 describes the preparation of a granular formulation of pranlukast that is characterized by dissolving sugar, a water-soluble polymer and/or a surfactant in purified water to lower the cohesiveness of pranlukast, subsequently suspending the pranlukast in the aqueous solution, and then spray drying the suspension product, which due to an improvement in the drug's surface properties, allows for easier formulation of a granular product. Following powder X-ray diffraction analysis, however, it is clearly seen that the cystallinity of the pranlukast is retained, and consequently, there is little improvement in the dissolution of pranlukast.

Pranlukast is availably commercially as ONON™ capsules, which contain pranlukast prepared by the WO 96/41628 process. This formulation exhibits a very low dissolution rate of less than 5%, at 37° C. and pH 6.8. (ONON™ capsules, prepared by the WO 96/41628 process are utilized herein in Experimental Examples 5 and 6.)

JP 8-73353 relates to liquid pharmaceutical preparations of pranlukast, such as eye drops, nasal drops or injections, all of which use polyvinylpyrrolidone or β-cyclodextrin as a solubilizer. WO 99/04790 also describes liquid preparations of pranlukast, such as an aqueous solution including a surfactant and a suspension including a water-soluble polymer. Although both of these preparations may be orally administered, due to the low dissolution of pranlukast, the usual individual dose of the solution will be several hundred milliliters (mL), making this formulation impractical. In the case where solubility of the pranlukast has been improved by controlling the pH of the solution, precipitation of the drug in the presence of gastric acid is seen. With regard to the suspension formulation, as the crystallinity of pranlukast is retained, one would not expect to see any substantial improvement in either the dissolution or the oral absorption rate of the pranlukast.

It is known that a solid dispersion formulation, which is frequently used as a method for improving the oral absorption of slightly soluble drugs, improves both the in vitro and the in vivo dissolution properties of the drug. A solid dispersion is a type of mixture where at least one active component is uniformly dispersed in a solid state polymer or an inert carrier and can be prepared by such methods as coprecipitation, coevaporation, freeze drying, spray drying, cogrinding and the like. J. Pharm. Sci., 82:32-38 (1993).

Examples of pharmaceutical compositions wherein the dissolution rate of the active drug has been improved with the use of a solid dispersion formulation, include, but are not limited to, a dicumarol-polyethyleneglycol solid dispersion, J. Pharm. Sci., 70:1353-7 (1981); a mefenamic acid-polyethyleneglycol solid dispersion, Pharm. Develop. Technol., 3:405-412 (1998); beads having a core coated with an antifungal and a polymer, Published International Patent Application WO 94/05263; a diazepam-polyethyleneglycol solid dispersion, Pharm. Acta. Helv., 64:90-93 (1989); a furosemide-polyvinylpyrrolidone solid dispersion, J. Pharm. Pharmacol., 41:73-78 (1989); a nifedipine-enteric coating agent solid dispersion, Chem. Pharm. Bull., 33:388-391 (1985); an enteric solid dispersion of cyclosporin, Chem. Pharm. Bull., 37:2542-2544 (1989); a tolbutamide-enteric coating polymers solid dispersion, Chem. Pharm. Bull., 35:3800-3806 (1987) and a triazole-based antifungal agent solid dispersion, Chem. Pharm. Bull., 44:568-571 (1996).

Unfortunately, however, converting a slightly soluble drug into a solid dispersion formulation does not always improve the dissolution rate following oral administration. In the above examples, for instance, the dissolution rate of any particular drug was raised no more than 85%. Moreover, with regard to the triazole-based antifungal agent solid dispersion MFB-1041 (Chem. Pharm.Bull., 44:568-71), which was formulated to contain hydroxypropylcellulose, hydroxypropylmethylcellulose, hydroxypropylmethylcellulose phthalate and carboxymethylethylcellulose, the dissolution rate was reported to be less than 12% and after 1 hour, only 6%.

With respect to the drug category of leukotriene antagonists, and in particular, pranlukast, the present inventor is not aware of any studies or reports that have described the preparation of a solid dispersion formulation as a means to improve the drugs' dissolution properties and oral absorption rates. In light of the absence of any such prior disclosures, the present inventor has conducted intensive studies and has arrived at a solid dispersion formulation of pranlukast that greatly improves the oral bioavailability of this only slightly water soluble drug.

### SUMMARY OF THE INVENTION

A main objective of the present invention lies in providing a therapeutically effective pharmaceutical composition of pranlukast for oral administration, as well as a method for its preparation and methods of treating such conditions as bronchial asthma and allergic rhinitis with an oral formulation of the composition. To accomplish this objective, the present inventor has discovered a formulation of pranlukast that exhibits vastly improved oral bioavailability of the drug, due to a significant improvement in the in vitro and in vivo dissolution properties of the drug.

Accordingly, the principles of the present invention provide a pharmaceutical composition of pranlukast, a drug known to have only minimal water solubility; a method for producing the composition; an oral formulation of the composition, and methods of treating bronchial asthma and allergic rhinitis with the composition.

More specifically, the principals of the present invention provide a solid dispersion formulation for oral administration, that comprises an amount of pranlukast dispersed in a polymer carrier comprising at least one polymer chosen from hydroxypropylcellulose (“HPC”) and hydroxypropylmethylcellulose (“HPMC”). According to the principles of the invention, the composition provides a formulation of pranlukast that exhibits vastly improved dissolution and bioavailability upon oral administration.

In order to raise the absorption rate of a slightly water soluble drug by improving its dissolution by way of a solid dispersion formulation, it is first important to determine what type of pharmaceutical carrier, and what active drug to carrier composition ratio will efficiently maintain a high dissolution level of the drug in the intestinal tract. Through his intensive studies, the present inventor has discovered a formulation that maintains a very high level of pranlukast dissolution in the intestinal tract, the active absorption site for the drug. The formulation accomplishes this advantage through the use of a polymer carrier.

Preferably, the weight ratio of polymer to pranlukast will be within the range of 0.25:1 to 5:1, and more preferably, within the range of 0.5:1 to 3:1. Where the ratio is too low, the dissolution of pranlukast will be greatly reduced, and where the ratio is increased beyond the preferred range, difficulties in the preparation of the solid dispersion formulation begin to appear. For example, when the solid dispersion formulation is formulated into such dosage forms as tablets, capsules, granules or dry syrups, a granulation step, such as dry granulation, will be required, and if the amount of polymer in the composition is too high, compression molding will be very difficult due to the cohesiveness of the polymer and, the disintegration of the resulting granules or tablets will be significantly curtailed and lead to retarded dissolution. To compensate for the impeded dissolution rate, the use of an excessive amount of diluents would be necessary, in order to lower the concentration of the polymer.

In another embodiment of the present invention, the composition further comprises an amount of hydroxypropylmethylcellulose phthalate 50 (“HPMC-50”). In this embodiment, the HPMC-50 is substituted as a part of the total weight of the polymer component, with the preferred weight ratio of HPMC-50 to polymer being within the range of 0.1:1 to 4:1, and more preferably, in the range of 0.25:1 to 2:1. When the weight ratio of HPMC-50 to polymer exceeds 4:1, the dissolution of pranlukast is greatly reduced.

As compared to the previously described solid dispersion formulation, this embodiment of the invention, wherein a portion of the polymer component is substituted with an amount of HPMC-50, exhibits faster disintegration, but an equal dissolution rate at 37° C. and pH 6.8.

Surprisingly and unexpectedly, the solid dispersion formulation of the invention exhibits a vast improvement in drug dissolution rate, shown to be at least 85% and often approaching 100%, and this improvement is directly reflected in real bioavailability.

A further aspect of the present invention provides a method for preparing a pranlukast solid dispersion formulation as herein described. The method comprises dissolving an amount of pranlukast and a solid state inert polymer carrier polymer in a liquid solvent and then drying the solvent according to any conventional drying method such as, for example, spray drying or vacuum drying, with a spray drier, a fluidized bed granulator, a CF granulator, a vacuum drier, or the like.

As pranlukast has a very low solubility in organic solvents, extensive studies with various organic solvents were required in order to determine the most effective solvent. As a result of these intensive studies, the present inventor discovered that pranlukast may be easily dissolved to a concentration of 1% or more, with the use of a dichloromethane (“DCM”)/methanol solvent mixture; heat may optionally be applied. A pranlukast concentration of at least 1% is preferred for the efficient mass production of the solid dispersion formulation.

While the methanol component might be substituted by ethanol, it is much more difficult to obtain a concentrated pranlukast solution in such a solvent as compared to the DCM/methanol solvent. Also, while a single solvent, such as, for example, DCM, methanol, other alcohols or a haloalkane, may also be used in the preparation of the formulation, such modifications once again make it much more difficult to prepare a solution having a concentration of pranlukast of at least a 1%.

Thus, a DCM/methanol solvent mixture is preferred, with the preferred volume ratio of DCM to methanol in the mixture being within the range of 2:1 to 5:1, and more preferably, within the range of 3:1 to 4.5:1.

While it is also possible to prepare the pranlukast solid dispersion formulation by melting the pranlukast and polymer carrier rather than dissolving them in a solvent, the melting temperature of pranlukast is very close to its decomposition temperature, making this method quite difficult.

The pranlukast solid dispersion formulation may further comprise such other components as surfactants, preservatives, complex-forming agents, electrolytes, discharging agents, as well as other active ingredients compatible with pranlukast, such as, for example, steroids, bronchodilators, antitussives, expectorants, mixtures thereof, and the like.

The pranlukast solid dispersion formulation of the present invention can be formulated into any conventional pharmaceutical dosage form, although oral dosage forms are preferred. Exemplary dosage forms include, but are not limited to, tablets, capsules, granules, dry syrups, eye drops, nasal sprays and formulations for inhalation.

The most preferred dosage form is an oral tablet, which may be manufactured using conventional components or diluents, as well as conventional tableting machines and techniques. The tablets may further include one or more additional components, such as, for example, diluents, disintegrator, binders, lubricants, gliding agents, coating agents, sweetening agents, flavoring agents and coloring agents and the like, some of which may be included for multiple purposes.

In addition, tablets prepared according to the present invention may be film-coated, for example, for flavoring, for convenience of intake, for improving drug stability, as well as, simply just for aesthetic reasons.

### BRIEF DESCRIPTION OF THE DRAWINGS

FIG. 1 illustrates the dissolution rate of a pranlukast solid dispersion formulation comprising polymer and pranlukast in a 1.5:1 weight ratio at a temperature of 37° C. and pH 6.8 (Examples 5 and 7; Comparative Examples 1-8);

FIG. 2 illustrates the dissolution rate of a pranlukast solid dispersion formulation comprising HPC and pranlukast in various weight ratios, at a temperature of 37° C. and pH 6.8 (Examples 1-6);

FIG. 3 illustrates the dissolution rate of a pranlukast solid dispersion formulation comprising polymer and pranlukast in a 1:1 weight ratio, wherein the polymer comprises various combinations of HPC and HPMC-50, at a temperature of 37° C. and pH 6.8 (Examples 8-12);

FIG. 4 illustrates the dissolution rate of a pranlukast solid dispersion formulation comprising polymer and pranlukast in various weight ratios, wherein the polymer comprises a 1:1 weight ratio combination of HPMC-50 and HPC, at a temperature of 37° C. and pH 6.8 (Examples 10 and 13-17);

FIG. 5 illustrates the dissolution rate of a single pranlukast tablet, prepared according to Example 18 (total weight of pranlukast: 120 mg), two commercial ONON™ capsules (total weight of pranlukast: 225 mg) and pranlukast bulk powder (total weight of pranlukast: 225 mg) at a temperature of 37° C. and pH 6.8;

FIG. 6 illustrates the plasma concentration of pranlukast achieved upon separate oral administration to six healthy adult males of either a single pranlukast tablet prepared according to Example 18 (total pranlukast administered: 120 mg) or two ONON™ capsules (total pranlukast administered: 225 mg); and

FIG. 7 illustrates the results of powder X-ray diffraction tests for:

| | |
|---|---|
| A | pranlukast crude powder; |
| B | commercial ONON ™ capsule filling; |
| C | HPC; |
| D | HPMC; |
| E | HPMC-50; |
| F | talc; |
| G | solid dispersion composition prepared according to |
| | Example 4; |
| H | solid dispersion composition prepared according to |
| | Example 7; and |
| I | solid dispersion composition prepared according to |
| | Example 10. |

### EXAMPLES

The following Examples illustrate various embodiments of the invention, as well as compare the dissolution properties, the in vivo bioavailability and the powder X-ray diffusion analysis test results, for these embodiments as to commercially available ONON™, and bulk pranlukast powder. These examples are for illustration only, and are not intended to limit the scope of the invention in any manner.

### Examples 1-7

### Preparation of Solid Dispersion Compositions

A solid dispersion composition as described in TABLE 1 was prepared according to the following method: 10 g Pranlukast and 1.25 g HPC (polymer) were mixed, followed by the addition of 200 mL methanol and 800 mL DCM, whereby the mixture was stirred and heated to 40° C. 0.5 g Talc was then added and the resulting product was dried under stirring in a spray drier set to a spraying speed of 70 mL/min. During the drying phase, the inlet air temperature was in the range of 95 to 105° C., the outlet air temperature was in the range of 40° to 50° C., and the jet pressure was controlled to be about 3 kg/cm².

Compositions according to Examples 2-7 of TABLE 1 were prepared according to the same method.

TABLE 1
Compositions of Examples 1-7

Polymer

| Example | Pranlukast | HPC | | HPMC | | Talc |
|---|---|---|---|---|---|---|
| 1 | 10 g | 1.25 | g | 0 | | 0.5 g |
| 2 | 10 g | 2.5 | g | 0 | | 0.5 g |
| 3 | 10 g | 5 | g | 0 | | 0.5 g |
| 4 | 10 g | 10 | g | 0 | | 0.5 g |
| 5 | 10 g | 15 | g | 0 | | 0.5 g |
| 6 | 10 g | 30 | g | 0 | | 0.5 g |
| 7 | 10 g | 0 | | 15 | g | 0.5 g |

### Comparative Examples 1 to 8

Solid dispersion formulations having the compositions set forth in TABLE 2, were prepared according to the method of Example 1.

TABLE 2
Compositions of Comparative Examples 1 to 8

| Comparative | | | | |
|---|---|---|---|---|
| Example | Pranlukast | Polymer | | Talc |
| 1 | 10 g | polyvinylpyrrolidone (PVP) | 15 g | 0.5 g |
| 2 | 10 g | polyethylene glycol (PEG 6000) | 15 g | 0.5 g |
| 3 | 10 g | HPMC-50 | 15 g | 0.5 g |
| 4 | 10 g | HPMC-55 | 15 g | 0.5 g |
| 5 | 10 g | ethylcellulose (EC) | 15 g | 0.5 g |
| 6 | 10 g | Poloxamer ® F-127 (PLX) | 15 g | 0.5 g |
| 7 | 10 g | Eudragit ® E (EUD-E) | 15 g | 0.5 g |
| 8 | 10 g | Eudragit ® L (EUD-L) | 15 g | 0.5 g |

### Examples 8 to 12

Solid dispersion formulations having the compositions set forth in TABLE 3, were prepared according to the method of Example 1. In these Examples, the weight ratio of total polymer to drug is 1:1, while the weight ratio between the two polymers differs.

TABLE 3
Compositions of Examples 8 to 12

Polymer

| Example | Pranlukast | HPC | HPMC-50 | Talc |
|---|---|---|---|---|
| 8 | 10 g | 8.00 | 2.00 | 0.5 g |
| 9 | 10 g | 6.67 | 3.33 | 0.5 g |
| 10 | 10 g | 5.00 | 5.00 | 0.5 g |
| 11 | 10 g | 3.33 | 6.67 | 0.5 g |
| 12 | 10 g | 2.00 | 8.00 | 0.5 g |

### Examples 13 to 17

Solid dispersion formulations having the compositions set forth in TABLE 4, were prepared according to the method of Example 1. In these Examples, the weight ratio between the two polymers is 1:1, while the weight ratio of polymer to drug differs.

TABLE 4
Compositions of Examples 13 to 17

Polymer

| Example | Pranlukast | HPC | HPMC-50 | Talc |
|---|---|---|---|---|
| 13 | 10 g | 20.00 | 20.00 | 0.5 g |
| 14 | 10 g | 10.00 | 10.00 | 0.5 g |
| 15 | 10 g | 7.50 | 7.50 | 0.5 g |
| 16 | 10 g | 2.50 | 2.5 | 0.5 g |
| 17 | 10 g | 1.25 | 1.25 | 0.5 g |

### Example 18

### Preparation of Tablets Using Solid Dispersion Composition

Tablets containing a 1:1 weight ratio of polymer to pranlukast and a 1:1 weight ratio polymer mixture of HPC and HPMC-50 (set forth as Example 10) were prepared according to the composition in TABLE 5.

TABLE 5
Composition of Tablets for Example 18

| Component | Weight/tablet | | Weight %/tablet |
|---|---|---|---|
| Tablet Core | | | |
| pranlukast | 120 | mg | 20.00 |
| HPC | 60 | mg | 10.00 |
| HPMC-50 | 60 | mg | 10.00 |
| talc | 6 | mg | 1.00 |
| lactose | 175 | mg | 29.17 |
| sodium chloride | 30 | mg | 5.00 |
| Premelose | 63 | mg | 10.50 |
| Compritol | 48 | mg | 8.00 |
| Aerosil | 5 | mg | 0.83 |
| Crospovidone | 18 | mg | 3.00 |
| Total for Table Core | 585 | mg | 97.50 |
| Film coating | | | |
| HPMC | 13 | mg | 2.17 |
| propyleneglycol | 2 | mg | 0.33 |
| Total for Film Coating | 15 | mg | 2.50 |
| TOTAL per TABLET | 600 | mg | 100.00 |

### Experimental Example 1—Dissolution Test I

### Type of Polymer

Dissolution profiles of the pranlukast solid dispersion compositions of Examples 5 and 7 and Comparative Examples 1 to 8 were compared. In each of these compositions, the weight ratio of polymer to drug was maintained at 1.5:1, and only the type of polymer was altered. The dissolution test was conducted according to the Method II Dissolution Test (Paddle method) set forth in the United States Pharmacopoeia.

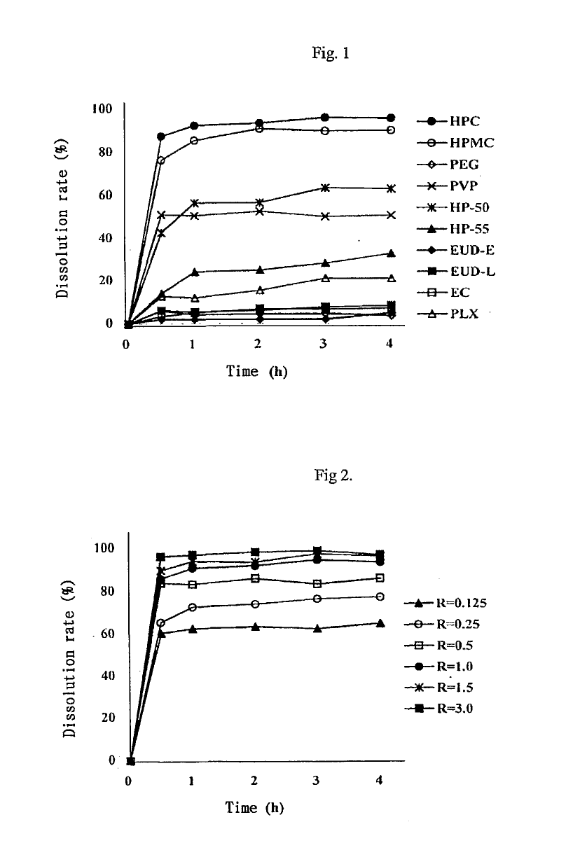

900 mL of the second fluid (pH 6.8) was added into the dissolution test device and a sample (according to one of the test Examples) providing 120 mg of pranlukast was added while maintaining the temperature at 37° C. Time-dependent dissolution was determined under stirring with a speed of 100 rpm. At each time-point, a 1 mL aliquot was taken up into a silicone-coated tube, centrifuged and the supernatant was subjected to HPLC analysis. The results of Dissolution test 1 for a sample according to each of Examples 5 and 7 and Comparative Examples 1 to 8 are shown in FIG. 1.

As is clearly shown in FIG. 1, while the pranlukast solid dispersions of Example 5 (polymer: HPC) and Example 7 (polymer: HPMC) exhibit dissolution rates of 85% or more, the compositions of Comparative Examples 1 to 8 (polymer: PVP, PEG 6000, HPMC-50, HPMC-55, EC, PLX, EUD-E and EUD-L, respectively), all exhibit dissolution rate of 65% or less.

### Experimental Example 2—Dissolution Test II

### Weight Ratio of HPC to Pranlukast

Dissolution profiles of the pranlukast solid dispersion compositions of Examples 1 to 6 were compared. The dissolution test was conducted as in Experimental Example 1. In each of these Examples, HPC was used as the polymer, although the weight ratio of HPC to drug was sequentially increased (0.125:1, 0.25:1, 0.5:1, 1:1, 1.5:1 and 3:1, respectively). The results are shown in FIG. 2.

While each of the solid dispersion compositions tested exhibited a relatively high dissolution rate, the compositions wherein the weight ratio of HPC to pranlukast was 0.5:1 or higher (Examples 3 to 6), exhibited very high dissolution rates of 85% or more.

### Experimental Example 3—Dissolution Test III

### Weight Ratio of HPMC-50 to HPC

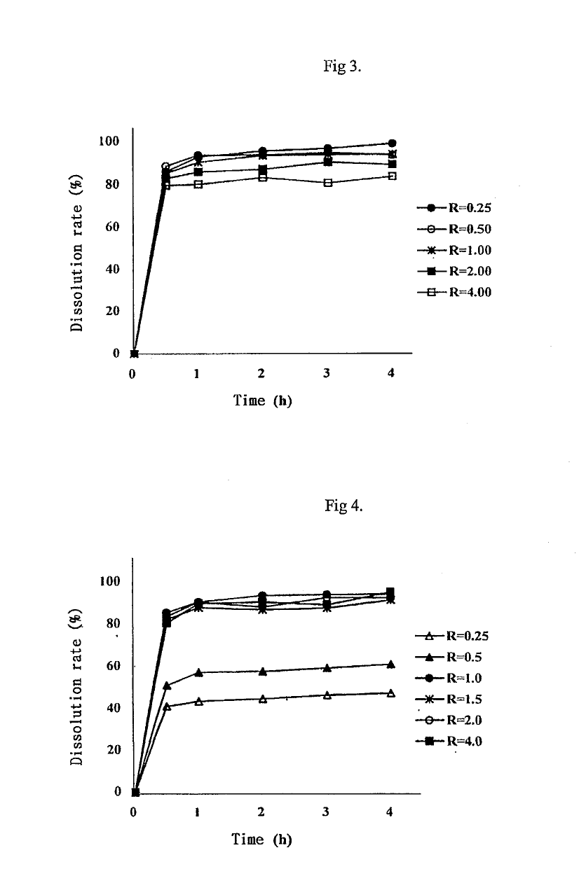

Dissolution profiles of the pranlukast solid dispersion compositions of Examples 8 to 12 were compared. The dissolution test was conducted as in Experimental Example 1. In each of these Examples, the weight ratio of total polymer to drug was maintained at 1:1, while the weight ratio of HPMC-50 to HPC was sequentially increased (0.25:1, 0.5:1, 1:1, 1.5:1, 2:1 and 4:1, respectively). The results are shown in FIG. 3.

In those Examples where the weight ratio of HPMC-50 to HPC was 1:1 or lower (Examples 8 to 10), there was no significant difference in the dissolution rate, but when the ratio was increased to 2:1 or higher (Examples 11 and 12), the dissolution rate decreased somewhat.

### Experimental Example 4—Dissolution Test IV

### Weight Ratio of Polymer (1:1 HPMC-50:HPC) to Pranlukast

Dissolution profiles of the pranlukast solid dispersion compositions of Examples 10 and 13-17 were compared. The dissolution test was conducted as in Experimental Example 1. In each of these Examples, the weight ratio of HPMC-50 to HPC was maintained at 1:1, while the weight ratio of the total weight of the polymer mixture to pranlukast was altered (1:1, 4:1, 2:1, 1.5:1, 0.5:1, 0.25:1, respectively). The results are shown in FIG. 4.

In those Examples where the weight ratio of total polymer to pranlukast was 1:1 or higher (Examples 10 and 13-15), there was no significant difference in the dissolution rate, but when the ratio was decreased to 0.5:1 or lower (Examples 16 and 17), the dissolution rate was noticeably declined.

### Experimental Example 5—Comparative Dissolution Test

A comparative dissolution test was conducted according to the Method II Dissolution Test (Paddle method) set forth in the U.S. Pharmacopoeia for one tablet prepared according to Example 18 (total weight of pranlukast: 120 mg), two commercial ONON™ capsules (total weight of pranlukast: 225 mg) and pranlukast bulk powder (total weight of pranlukast: 225 mg).

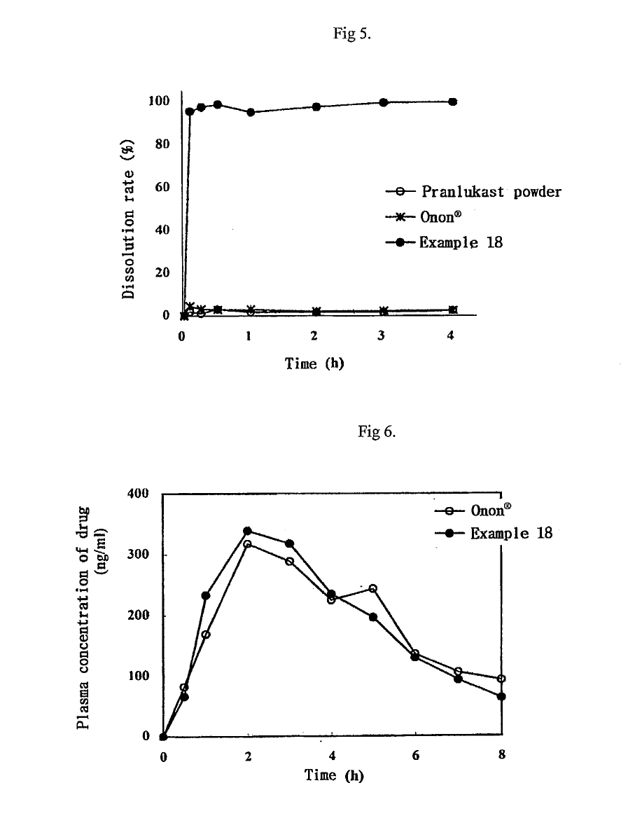

The results are shown in FIG. 5. While the dissolution rates of pranlukast bulk powder and the commercial ONON™ preparation were less than 5%, the dissolution rate of the pranlukast tablet prepared according to the present invention was 85% or more.

### Experimental Example 6—In vivo Availability Test

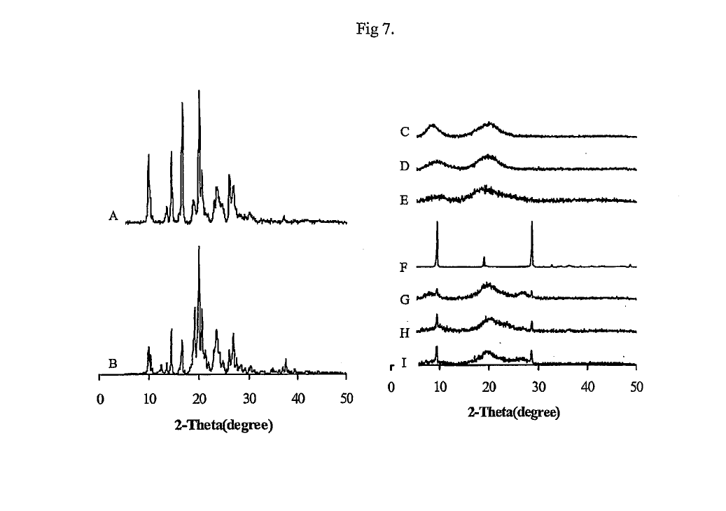

A single pranlukast tablet prepared according to Example 18 (total pranlukast administered: 120 mg) and two commercial ONON™ capsules (total pranlukast administered: 225 mg), were cross-administered orally to 6 healthy adult males in a fasting state. The plasma concentration of pranlukast compared. Results are shown in FIG. 6.

Upon the administration of the single 120 mg pranlukast tablet prepared according to Example 18, the plasma concentration of pranlukast was similar to that observed following the administration of two 112.5 mg ONON™ capsules. In addition, the area under the plasma concentration-time curve from time 0 to time 8 hours was also similar—1554 ng.h/mL for the test tablet and 1534 ng.h/mL for the ONON™ dose. These results clearly show the benefit provided by the solid dispersion formulation of the present invention, in that even with the administration of half the amount of pranlukast, the test table provides equal efficacy, as shown by the high in vivo bioavailability.

### Experimental Example 7—Powder X-ray Diffraction Analysis

To evaluate the presence of crystallinity of pranlukast within the compositions of the present invention, powder X-ray diffraction analysis was carried out. Testing was conducted at room temperature with a DMAX/1200 X-ray diffraction analyzer (Rikaku Denki, Tokyo, Japan) (CuKa, 40 kV, 20 mA, 5 degree/min).

The results are illustrated in FIG. 7, wherein “A” represents pranlukast crude powder; “B” represents commercial ONON™ capsule filling; “C” represents HPC; “D” represents HPMC; “E” represents HPMC-50; “F” represents talc; “G” represents a solid dispersion formulation prepared according to Example 4; “H” represents a solid dispersion formulation prepared according to Example 7; and “I” represents a solid dispersion formulation prepared according to Example 10.

Referring to FIG. 7, crystalline peaks are clearly seen for A and B, confirming the presence of crystallinity within the test samples; in clear contrast no such peaks are seen for test samples G, H or I, each representing a solid dispersion composition of the present invention. As there is no evidence of crystallinity in the samples, the test confirms that the compositions of the invention are in the form of a solid dispersion, wherein the pranlukast exists in amorphous form. The powder X-ray diffraction analysis for polymers C, D and E, as well as for talc are provided solely for reference.

Clearly, the formulation of the present invention remarkably improves the oral bioavailability of pranlukast, by improving both the drug's in vitro and in vivo dissolution, thus making it very desirable for use in preparing pranlukast for oral administration, and providing both economic and therapeutic benefits.

This invention has been described in terms of specific detailed embodiments, which are presented by way of illustration only, and the invention is not intended to be limited therto. Modifications and variations within the spirit and scope of the claims that follow will be readily apparent from this disclosure, as those skilled in the art will appreciate.

We claim:

1. A pharmaceutical composition comprising an amount of pranlukast (4-oxo-8-[4-(4-phenylbutoxy)benzoyl-amino]-2-(tetrazol-5-yl)-4H-1-benzopyran hemihydrate) uniformly dispersed in a solid state inert polymer carrier comprising at least one of hydroxypropylcellulose (“HPC”) or hydroxypropylmethylcellulose (“HPMC”).

2. The pharmaceutical composition of claim 1, wherein the weight ratio of the polymer carrier to pranlukast is within a range of 0.25:1 to 5:1.

3. The pharmaceutical composition of claim 2, wherein the weight ratio of the polymer carrier to pranlukast is within a range of 0.5:1 to 3:1.

4. The pharmaceutical composition of claim 1, further comprising an amount of hydroxypropylmethylcellulose phthalate 50 (“HPMC-50”).

5. The pharmaceutical composition of claim 4, wherein the weight ratio of HPMC-50 to the at least one of HPC and HPMC is within a range of 0.1:1 to 4:1.

6. The pharmaceutical composition of claim 5, wherein the weight ratio of HPMC-50 to the at least one of HPC and HPMC is within a range of 0.25:1 to 2:1.

7. The pharmaceutical composition of claim 1, formulated for oral administration.

8. The pharmaceutical composition of claim 7, formulated as tablets, capsules, granules or a dry syrup.

9. A solid dispersion formulation for oral administration consisting essentially of a pharmaceutical composition comprising an amount pranlukast uniformly dispersed in a solid state inert polymer carrier comprising at least one of HPC or HPMC.

10. The solid dispersion formulation for oral administration of claim 9, wherein the weight ratio of the polymer carrier to pranlukast is within a range of 0.25:1 to 5:1.

11. The solid dispersion formulation for oral administration of claim 9, wherein the pharmaceutical composition further comprises an amount of HPMC-50.

12. The solid dispersion formulation for oral administration of claim 11, wherein the weight ratio of HPMC-50 to the at least one of HPC and HPMC is within a range of 0.1:1 to 4:1.

13. The solid dispersion formulation for oral administration of claim 9, formulated as tablets, capsules, granules or a dry syrup.

14. The solid dispersion formulation for oral administration of claim 9, further comprising one or more additional components chosen from surfactants, preservatives, complex-forming agents, electrolytes, discharging agents and other active ingredients compatible with pranlukast.

15. The solid dispersion formulation for oral administration of claim 14, wherein the one or more additional components is chosen from steroids, bronchodilators, antitussives and expectorants.

16. A method of treating bronchial asthma in a patient, comprising administering to a patient in need of treatment for bronchial asthma, a therapeutically effective dosage of a solid dispersion formulation according to claim 9.

17. A method of treating allergic rhinitis in a patient, comprising administering to a patient in need of treatment for allergic rhinitis, a therapeutically effective dosage of a solid dispersion formulation according to claim 9.

18. A method for preparing a solid dispersion formulation consisting essentially of a pharmaceutical composition comprising pranlukast uniformly dispersed in a solid state inert polymer carrier comprising at least one of HPC or HPMC, the method comprising:
a) dissolving the pranlukast and the solid state inert polymer carrier in a liquid solvent mixture of dichloromethane (“DCM”) and methanol (“MeOH”); and
b) drying the solvent to obtain a solid dispersion of pranlukast in the polymer carrier.

19. The method of claim 18, wherein the volume ratio of DCM:MeOH in the liquid solvent mixture is within a range of 2:1 to 5:1.

20. The method of claim 19, wherein the volume ratio of DCM:MeOH in the liquid solvent mixture is within a range of 3:1 to 4.5:1.
